(19) United States
(12) Patent Application Publication
(10) Pub. No.: US 2020/0354304 A1
(43) Pub. Date: Nov. 12, 2020

(54) PROCESS FOR THE TREATMENT OF A PHOSPHITE-CONTAINING WASTE STREAM

(71) Applicant: NOURYON CHEMICALS INTERNATIONAL B.V., ARNHEM (NL)

(72) Inventors: Bjorn TER HORST, Maarn (NL); Chantal KLERK, Elst (NL); Paulus Johannes Maria DIJKGRAAF, Holten (NL); Michel VAN DEN BERG, Elst (NL)

(73) Assignee: NOURYON CHEMICALS INTERNATIONAL B.V., ARNHEM (NL)

(21) Appl. No.: 16/765,917

(22) PCT Filed: Nov. 20, 2018
(86) PCT No.: PCT/EP2018/081885

§ 371 Date: May 21, 2020

(30) Foreign Application Priority Data
Nov. 23, 2017 (EP) 17203316.9

(51) Int. Cl. C07C 67/60; C01B 25/45; C07F 9/08; C02F 9/00

(52) U.S. Cl. CPC C07C 67/60; C01B 25/451; C02F 1/66; C02F 9/00; C07F 9/08

(57) ABSTRACT

Process for the treatment of a phosphite-containing waste stream, said process comprising the following steps: (a) optionally neutralizing the waste stream to a pH in the range 6.0-8.0, (b) adding the following compounds to the waste stream in any order of addition: (i) an oxidizing compound in order to oxidize said phosphite towards phosphate, (ii) an NH₄⁺ source, (iii) a Mg²⁺ source, thereby forming a precipitate, (c) followed by isolating the precipitate from the waste stream, wherein the process is conducted under atmospheric pressure and at a temperature not exceeding 90° C.

### CROSS-REFERENCE TO RELATED APPLICATION

This application is a U.S. National-Stage entry under 35 U.S.C. § 371 based on International Application No. PCT/EP2018/081885, filed Nov. 20, 2018, which was published under PCT Article 21(2) and which claims priority to European Application No. 17203316.9, filed Nov. 23, 2017, which are all hereby incorporated in their entirety by reference.

### TECHNICAL FIELD

In the production of acid chlorides and organic peroxides, in particular diacyl peroxides and peroxyesters, waste streams are created that contain large amounts of phosphorous-containing compounds, including phosphites and phosphates.

### BACKGROUND

In order for such waste streams to be treated in a biological waste water treatment unit, the concentration of these phosphorous-containing compounds has to be reduced significantly. The object of the present invention therefore relates to removal of these compounds from waste streams, in such a way that a valuable product is obtained.

This object has been met by providing a process that results in the production of struvite. Struvite is a slow release crystal fertilizer that has the potential to help reduce the long-term threat to food security from the declining reserves of phosphate rock.

In pure form, struvite has the formula MgNH4PO4.6H2O and can be obtained by precipitating magnesium, ammonium, and phosphate in a 1:1:1 molar ratio. However, the term “struvite” is also used for materials that do not solely consist of pure struvite. Also materials that contain minerals like magnesium phosphates in addition to MgNH4PO4.6H2O and Mg—NH4-PO4 precipitates with a different stoichiometry are generally called struvite.

Examples of such precipitates with a different stoichiometry include schertelite [(NH4)2H2Mg(PO4)2.4H2O] and hannayite [(NH4)2H4Mg3(PO4)4.8H2O].

This variation in composition does not limit the application of struvite as a fertilizer, as long as the user is aware of the actual mineral composition.

In this specification, the term “struvite” refers to a precipitate of magnesium, ammonium, and phosphate ions, irrespective of its precise stoichiometry and mineral structure.

It is known to produce struvite from waste streams, including municipal and agricultural effluents. The resulting struvite, however, may contain various amounts of contaminants, such as hormones, drug residues, pathogens, biocides, and crop protection products. Such contaminants make it unacceptance in agricultural applications.

Furthermore, known methods for producing struvite from waste streams require electrolysis of phosphites (JP2010-179214 A) or high pressure equipment (CN 102344209 A). In addition, other objects, desirable features and characteristics will become apparent from the subsequent summary and detailed description, and the appended claims, taken in conjunction with the accompanying drawings and this background.

### SUMMARY

The object of the present invention is therefore the provision of a process for the reduction of phosphorous in phosphite-containing waste streams, in particular waste streams from acid chloride and/or organic peroxide production, and at the same time producing struvite that is free of hormones, drug residues, pathogens, biocides, or crop protection products. A further object is the provision of a process that can be performed under atmospheric conditions and does not require the use of electrolysis equipment or of high pressure equipment such as autoclaves.

These objects are achieved by the process of the present invention, in which a phosphite-containing waste stream is treated according to the following steps:

a) optionally neutralizing the waste stream to a pH in the range 6.0-8.0,
b) adding the following compounds to the waste stream in any order of addition:
an oxidizing compound in order to oxidize said phosphite towards phosphate,
an NH₄⁺ source,
a Mg²⁺ source,
thereby forming a precipitate,
   c) followed by isolating the precipitate from the waste stream, wherein the process is conducted under atmospheric pressure and at a temperature not exceeding 90° C.

### DESCRIPTION

The following detailed description is merely exemplary in nature and is not intended to limit the invention or the application and uses of the invention. Furthermore, there is no intention to be bound by any theory presented in the preceding background of the invention or the following detailed description.

Examples of waste streams containing phosphite include scrubber waste water from acid chloride production and destruction waste water and filtrate wash water of organic peroxide production, in particular diacyl peroxide and/or peroxyester production.

Diacyl peroxides are generally prepared by reacting an acid chloride with H2O2. Peroxyesters are generally prepared by reacting an acid chloride with an organic hydroperoxide.

The acid chloride used in these processes is conventionally prepared by reacting a carboxylic acid with PCl₃. Excess PCl₃ is generally collected in a scrubber as H₃PO₃.

During the peroxydation reaction, part of the PCl₃ present in the crude acid chloride will be oxidized with H₂O₂, peroxy acids, and/or O₂ to POCl₃. After quenching with water, the POCl₃ reacts with H₂O to form PO₄³⁻ (H₃PO₄). Any remaining PCl₃ reacts with water under the formation of PO₃³⁻ (H₃PO₃).

In a preferred embodiment, at least part of the waste stream to be treated in the process of the present invention originates from an acid chloride production unit and/or from an organic peroxide production unit.

In one such preferred embodiment, at least part of the waste stream to be treated in the process of the present invention originates from an isobutyryl chloride, n-butyryl chloride, neopentanoyl chloride (pivaloyl chloride), n-pentanoyl chloride (valeroyl chloride), hexanoyl chloride, octanoyl chloride, nonanoyl chloride, neodecanoyl chloride, and/or lauroyl chloride production unit. Even more preferably, at least part of the waste stream to be treated in the process of the present invention originates from a lauroyl chloride, an n-decanoyl chloride, and/or a neodecanoyl chloride production unit.

Alternatively or additionally, at least part of the waste stream to be treated in the process of the present invention originates from a diacyl peroxide production unit and/or a peroxyester production unit.

Examples of such diacyl peroxides are di-isobutyryl peroxide, di-n-butyryl peroxide, di-n-pentanoyl peroxide (di-valeroyl peroxide), di-hexanoyl peroxide, di-octanoyl peroxide, di-nonanoyl peroxide, di-decanoyl peroxide, and di-lauroyl peroxide.

Examples of such peroxyesters are cumyl peroxyneodecanoate, 1,1,3,3-tetramethylbutyl peroxyneodecanoate, cumyl peroxyneoheptanoate, tert-amyl peroxyneodecanoate, tert-butyl peroxyneodecanoate, 1,1,3,3-tetramethylbutyl peroxypivalate, tert-butyl peroxyneoheptanoate, tert-amyl peroxypivalate, tert-butyl peroxypivalate, 2,5-dimethyl-2,5-di(2-ethylhexanoylperoxy)hexane, 1,1,3,3-tetramethylbutyl peroxy-2-ethylhexanoate, tert-amyl peroxy-2-ethylhexanoate, tert-butyl peroxy-2-ethylhexanoate, tert-butyl peroxydiehtlyacetate, tert-butyl peroxyisobutyrate, tert-amylperoxy acetate, tert-butyl peroxy-3,5,5-trimethylhexanoate, tert-amyl peroxybenzoate, tert-butyl peroxyacetate, and tert-butyl peroxybenzoate.

The phosphite-containing waste stream to be treated in the process according to the present invention preferably has a P-content of at least 10 ppm, more preferably at least 20 ppm, even more preferably at least 30 ppm, and most preferably at least 40 ppm. These contents can be determined by ICP.

It is preferred to first provide a waste stream with a pH in the range 6.0-8.0. This is especially preferred if hypochlorite is used as oxidizing compound in the next step, in order to prevent chlorine formation. Therefore, an optional first step in the process is neutralization of the waste stream to a pH in the range 6.0-8.0, preferably 6.5-7.5. Assuming that the waste stream will generally be of acidic nature, this neutralization involves the addition of base. Although any kind of base can be added, it is most preferred to add an alkali metal hydroxide, more preferably NaOH.

Should the waste stream contain any hydrogen peroxide, e.g. resulting from an organic peroxide production process, a reductor is preferably added to the neutralized waste stream in order to reduce such hydrogen peroxide.

Suitable reductors are sulfites, such as sodium sulfite, sodium bisulfite, sodium metabisulfite, and sodium sulfide.

In step b), an oxidizing compound is added to the waste stream in order to oxidize the phosphite (PO₃³⁻) towards phosphate (PO₄³⁻). An oxidizing compound is a chemical compound that can oxidize phosphite towards phosphate by a chemical oxidation reaction.

Examples of suitable oxidizing compounds are sodium hypochlorite, persulphate, perchlorate, ozone, the combination of ozone and H₂O₂, and ClO₂. Sodium hypochlorite is the preferred oxidizing compound, since it is able to oxidize phosphite to phosphate almost instantaneously.

The oxidation reaction can be monitored by a redox electrode. If the oxidizing compound is added slowly (e.g. dropwise), a steady potential—i.e. the absence of a decrease in potential directly after addition of the oxidizing compound—indicates complete oxidation towards phosphate.

Any excess of oxidizing compound can be reduced by the addition of a reductor, since the oxidizing compound (e.g. NaOCl) can potentially destroy the biological sludge in a water treatment unit.

Suitable reductors are sulfites, such as sodium sulfite, sodium bisulfite, sodium meta bisulfite, and sodium sulfide.

To the waste stream are also added an NH₄⁺ source and a Mg²⁺ source, in order to form struvite as a precipitate.

The NH₄⁺ source is preferably added in a molar ratio of nitrogen atoms relative to phosphorous atoms in the waste stream of 0.5-5.0, more preferably 0.5-2.0, even more preferably 0.5-1.0, and preferably 0.6-0.8. Higher amounts do not promote further P-removal from the waste stream, but do increase the N-content of the waste stream, which is evidently undesired from an environmental point of view.

The ammonium source is preferably gaseous ammonia, aqueous ammonia solution (ammonium hydroxide), or an ammonium salt. The ammonium salt is preferably selected from ammonium chloride, ammonium nitrate, ammonium sulphate, and ammonium bromide, sodium ammonium sulphate. More preferably, an ammonium salt is used instead of ammonia since commercial ammonia is not very consistent in concentration and because of its safety hazards and smell.

Most preferably, the ammonium source is ammonium chloride.

The Mg²⁺ source is preferably added in a molar ratio of magnesium atoms relative to phosphorous atoms in the waste stream of 1.0-3.0, more preferably 1.0-2.0, even more preferably 1.2-1.6, most preferably 1.3-1.4. The amount of phosphorous in the waste stream can be determined with ICP. Such an excess of Mg was found to improve the decrease in P-content of the waste stream, probably due to a reaction competing with MgNH₄PO₄. 6H2O formation (probably magnesium phosphate formation).

The magnesium source is preferably magnesium oxide or a magnesium salt selected from magnesium chloride, magnesium sulphate, magnesium hydroxide, and magnesium bromide. Most preferably, the magnesium source is magnesium chloride.

Although pure struvite contains Mg and NH4 in a 1:1 ratio, it is preferred to add the Mg2+ ions, relative to NH4+ ions, in a molar ratio in the range 1.0-2.5, more preferably 1.5-2.0. Such an excess of Mg source was found to improve the decrease in P-content of the waste stream, probably due to a reaction competing with MgNH₄PO₄.6H₂O formation (probably magnesium phosphate formation).

The ammonium source, the magnesium source, and the oxidizing compound may be added to the waste stream simultaneously or consecutively in any order.

It is however preferred to add the oxidizing compound before the addition of the ammonium source. More preferably, after addition of the oxidizing compound and the consequential oxidation of phosphite to phosphate, a reductor is added to reduce any remaining oxidizing compound, before the ammonium source is added. This is especially preferred if the oxidizing compound is a hypochlorite, in order to prevent evolution of toxic gases (such as chlorine and/or chloroamines) resulting from a reaction between hypochlorite and ammonium.

The magnesium source can be added simultaneously with the oxidizing compound or at a later stage. It is preferred to add both the magnesium and the ammonium source after the oxidation of phosphite to phosphate and the optional reduction of the oxidizing compound, because otherwise magnesium phosphates are formed, which negatively affect the layer thickness of the precipitate.

One embodiment of the invention therefore relates to a process comprising the following steps:
a) optionally neutralizing the phosphite-containing waste stream to a pH in the range 6.0-8.0,
b1) adding a Mg²⁺ source and an oxidizing compound to the phosphite-containing waste stream in order to oxidize said phosphite towards phosphate,
b2) optionally adding a reductor to the waste stream in order to reduce the oxidizing compound, followed by
b3) adding an NH₄⁺ source to the waste stream, thereby forming a precipitate, and
c) isolating the precipitate from the waste stream,
   wherein the process is conducted under atmospheric pressure and at a temperature not exceeding 90° C.

Another, more preferred embodiment of the invention relates to a process comprising the following steps:
a) optionally neutralizing the phosphite-containing waste stream to a pH in the range 6.0-8.0,
b1) adding an oxidizing compound to the phosphite-containing waste stream in order to oxidize said phosphite towards phosphate,
b2) optionally adding a reductor to the waste stream in order to reduce the oxidizing compound, followed by
b3) adding an NH₄⁺ source and a Mg²⁺ source to the waste stream, thereby forming a precipitate, and
c) isolating the precipitate from the waste stream,
   wherein the process is conducted under atmospheric pressure and at a temperature not exceeding 90° C.

In the latter embodiment, the ammonium and magnesium sources can be added to the waste stream as a pre-mix or individually in any order. It is, however, preferred to add them individually, because pre-mixing the two sources results in the formation of solid particles. Even more preferably, the NH₄⁺ source is added prior to the Mg²⁺ source in order to prevent magnesium phosphate formation prior to the addition of the ammonium source.

For practical reasons, the NH4⁺ source and the Mg²⁺ source are preferably added as aqueous solutions.

During step b), the waste stream is preferably stirred with a power input of 0.1-3.0 kW/m³, more preferably 0.1-2.0 kW/m³, and most preferably 0.3-1.0 kW/m³.

Lower stirring power results in the formation of larger precipitate particles; higher stirring power result in smaller precipitate particles. If the particles become too small, their settling speed decreases and settling times may become undesirably long.

Furthermore, it was found that higher stirring power increases the P-uptake; that is: it contributes to a further reduction of the P-content of the waste stream. It is theorized that the formation of smaller particles at higher stirring power results in better growing nuclei for struvite.

It is desired that at the end of step b), the pH of the waste stream is in the range 8-11, more preferably 10-11. At higher pH, the filterability of the precipitate is reduced and struvite formation may compete with Mg(OH)₂ formation. At lower pH, struvite tends to dissolve.

This means that, in a preferred embodiment, the pH is raised during step b) by the addition of a base (e.g. sodium hydroxide).

The process is conducted at atmospheric pressure and at a temperature not exceeding 90° C. The temperature is preferably not exceeding 70° C., more preferably not exceeding 50° C., and most preferably ambient temperature. Heating equipment may be applied during the process, but since the optional neutralization step a) and the optional pH raise in step b) are very exothermic, cooling equipment in order to prevent overheating might be more applicable.

The process according to the present invention can be conducted in any type of equipment that would appear suitable. Preferably, it is conducted in a stirred vessel.

Struvite precipitates from the waste stream and can be isolated in any suitable way. For instance, it can be settled in a settling pit or it can be filtered or centrifuged from the waste stream.

After isolation of struvite from the waste stream, the resulting stream preferably has a phosphorous content of less than 100 ppm, more preferably less than 50 ppm, and most preferably less than 10 ppm, as measured by ICP. This stream can be fed to a biological waste water treatment unit.

### EXAMPLES

### Example 1

Scrubber water (25 kg) originating from the production of lauroyl chloride from PCl3 and lauric acid, having a pH in the range 0-2 and containing approximately 3200 ppm P (determined with ICP) was charged to a 30 litre reactor equipped with mechanical agitator, a pH probe, and a redox electrode. Throughout the experiment, a power input of 0.2 kW/m3 was maintained. The scrubber water was brought to pH=7 with a 25% NaOH aqueous solution (6.3 kg).

Subsequently, the PO₃³⁻ present in the scrubber water was oxidized to PO₄³⁻ by addition of 1.656 kg of a 1.63 mmol/gram NaOCl solution. The oxidation was monitored with the redox electrode. Addition was continued in a drop-wise manner until a steady potential was observed (i.e. no decrease in potential directly after addition of NaOCl).

In a next step, excess NaOCl was reduced by the addition of 31 gram of an aqueous 1.89 mmol/gram sodium sulfite solution. This step was monitored by the redox electrode. A sharp drop in potential was observed.

A 25 wt % NH4Cl solution (0.740 kg; 1.3 molar equivalents NH4Cl based on P) was added to the resulting solution. This was followed by the addition of 1.097 kg of a 30 wt % MgCl2 solution (1.3 molar equivalents MgCl2 based on P).

Formation of small precipitate particles was observed instantaneously and stirring was continued for 2 minutes during which the pH was increased to 10.5 by addition of NaOH (25 wt %, 0.675 kg). At this pH, the precipitate was less soluble. The reactor volume was drained to a settling pit with overflow in which the precipitate particles settled at the bottom and a clear water stream containing 9.8 ppm phosphorous (determined by ICP) overflowed the settling pit.

### Example 2

Example 1 was repeated with different amounts of Mg. The results are presented in the Table below:

| equiv. | equiv. | Starting | Final P | Final N | Final Mg |
|---|---|---|---|---|---|
| Mg | N | P (ppm) | (ppm) | (ppm) | (ppm) |
| 0.7 | 0.7 | 3200 | 1300 | 320 | <1 |
| 0.8 | 0.8 | 3200 | 1000 | 380 | <1 |
| 0.9 | 0.9 | 3200 | 810 | 490 | <1 |
| 1.0 | 1.0 | 3200 | 620 | 440 | <1 |
| 1.1 | 1.1 | 3200 | 400 | 540 | <1 |
| 1.2 | 1.2 | 3200 | 190 | 600 | 1 |
| 1.3 | 1.3 | 3200 | 9.8 | 615 | 18 |

Until up to 1.2 equivalents of magnesium, almost all of the magnesium was consumed and the P-content dropped in an almost linear fashion. At 1.3 equivalents Mg, the concentration of magnesium in the remaining eluent started to increase and the P-content started to level off.

In addition, the final nitrogen content was relatively high, which suggests that the formed particles do not precisely react in a 1:1:1 ratio.

### Example 3

Example 1 was repeated with different amounts of Mg and with 0.7 (instead of 1.3) molar equivalents NH₄Cl. The results are presented in the Table below:

| equiv. | equiv. | Starting | Final P | Final N | Final Mg |
|---|---|---|---|---|---|
| Mg | N | P (ppm) | (ppm) | (ppm) | (ppm) |
| 1.2 | 0.7 | 3200 | 180 | 170 | 2 |
| 1.3 | 0.7 | 3200 | 23 | 130 | 33 |
| 1.4 | 0.7 | 3200 | 6 | 120 | 155 |
| 1.5 | 0.7 | 3200 | 4 | 120 | 215 |

### Example 4

Example 2 was repeated using 1000 grams waste water from the production of dilauroyl peroxide; said waste water contained 385 ppm P. Excess H₂O₂ in the wastewater was first destroyed by adding 3.7 gram sodium sulfite aqueous solution (1.89 mmol/gram).

The PO₃³⁻ present in the wastewater was oxidized to PO₄³⁻ by addition of 8.4 grams of NaOCl solution (1.63 mmol/gram solution). Next, 0.62 gram sodium sulfite aqueous solution (1.89 mmol/gram solution) was added to destroy excess NaOCl.

The amount of phosphite oxidized during this procedure was calculated as the amount of hypochlorite used in the oxidation step minus the amount of bisulfite added to destroy any excess of NaOCl, and amounted 12.52 mmol.

5 molar equivalents NH4Cl—based on oxidized phosphite—were added to the wastewater. The resulting mixture was divided in ten equal samples and incremental amounts of MgSO4 were added based on the total phosphate concentration

| equiv. | equiv. | Starting | Final P | Final N | Final Mg |
|---|---|---|---|---|---|
| Mg | N | P (ppm) | (ppm) | (ppm) | (ppm) |
| 0.7 | 5 | 385 | 190 | nd | 7 |
| 0.8 | 5 | 385 | 160 | nd | 5 |
| 0.9 | 5 | 385 | 135 | nd | 6 |
| 1.0 | 5 | 385 | 100 | nd | 8 |
| 1.1 | 5 | 385 | 80 | nd | 9 |
| 1.2 | 5 | 385 | 56 | nd | 11 |
| 1.3 | 5 | 385 | 42 | nd | 21 |

nd = not determined

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1-15. (canceled)

16. A process for treating a phosphite-containing waste stream, the process comprising the following steps:
a) optionally neutralizing the waste stream to a pH in the range 6.0-8.0,
b) adding the following compounds to the waste stream in any order of addition:
an oxidizing compound in order to oxidize the phosphite towards phosphate,
an NH4⁺ source,
a Mg²⁺ source,
thereby forming a precipitate
c) followed by isolating the precipitate from the waste stream,
wherein the process is conducted under atmospheric pressure and at a temperature not exceeding 90° C.

17. The process according to claim 16, wherein step b) comprises
b1) adding an oxidizing compound to the phosphite-containing waste stream in order to oxidize said phosphite towards phosphate,
b2) optionally adding a reductor to the waste stream in order to reduce the oxidizing compound, followed by
b3) adding an NH4⁺ source and a Mg²⁺ source to the waste stream, thereby forming a precipitate, and
c) isolating the precipitate from the waste stream,
wherein the process is conducted under atmospheric pressure and at a temperature not exceeding 90° C.

18. The process according to claim 16, wherein step b) comprises
b1) adding a Mg²⁺ source and an oxidizing compound to the phosphite-containing waste stream in order to oxidize said phosphite towards phosphate,
b2) optionally adding a reductor to the waste stream in order to reduce the oxidizing compound, followed by
b3) adding an NH₄⁺ source to the waste stream, thereby forming a precipitate, and
c) isolating the precipitate from the waste stream,
wherein the process is conducted under atmospheric pressure and at a temperature not exceeding 90° C.

19. The process according claim 16, wherein the NH₄⁺ source is added in a molar ratio of nitrogen atoms relative to phosphorous atoms in the waste stream of 0.5-1.0.

20. The process according to claim 15, wherein the Mg²⁺ source is added in a molar ratio of magnesium atoms relative to phosphorous atoms in the waste stream of 1.0-2.0.

21. The process according to claim 16, wherein the oxidizing compound comprises sodium hypochlorite.

22. The process according to claim 16, wherein the waste stream at the end of step b) has a pH in the range 8-11.

23. The process according to claim 17, wherein during step b3) the NH₄⁺ source is added prior to the Mg²⁺ source.

24. The process according to claim 16, wherein the NH₄⁺ source is selected from gaseous ammonia, aqueous ammonia solution (ammonium hydroxide), and ammonium salts.

25. The process according to claim 16, wherein the Mg²⁺ source is selected from magnesium chloride, magnesium sulphate, magnesium hydroxide, magnesium bromide, and magnesium oxide.

26. The process according to claim 16, wherein the waste stream contains hydrogen peroxide and wherein, between steps a) and b), a reductor is added to the neutralized waste stream in order to reduce the hydrogen peroxide.

27. The process according to claim 16, wherein at least part of the waste stream is the effluent from an acid chloride production process.

28. The process according to claim 27, wherein the waste stream is from the production of isobutyryl chloride, n-butyryl chloride, neopentanoyl chloride (pivaloyl cloride), n-pentanoyl chloride (valeroyl chloride), hexanoyl chloride, octanoyl chloride, nonanoyl chloride, neodecanoyl chloride, or lauroyl chloride.

29. The process according to claim 16, wherein at least part of the waste stream results from a diacyl peroxide production process.

30. The process according to claim 29, wherein the diacyl peroxide is selected from the group consisting of di-isobutyryl peroxide, di-n-butyryl peroxide, di-neopentanoyl peroxide (di-pivaloyl peroxide), di-n-pentanoyl peroxide (di-valeroyl peroxide), di-hexanoyl peroxide, di-octanoyl peroxide, di-nonanoyl peroxide, di-neodecanoyl peroxide, and di-lauroyl peroxide.

31. The process according to claim 16, wherein at least part of the waste stream results from a peroxyester production process.

32. The process according to claim 31, wherein the peroxyester is selected from the group consisting of cumyl peroxyneodecanoate, 1,1,3,3-tetramethylbutyl peroxyneodecanoate, cumyl peroxyneoheptanoate, tert-amyl peroxyneodecanoate, tert-butyl peroxyneodecanoate, 1,1,3,3-tetramethylbutyl peroxypivalate, tert-butyl peroxyneoheptanoate, tert-amyl peroxypivalate, tert-butyl peroxypivalate, 2,5-dimethyl-2,5-di(2-ethylhexanoylperoxy)hexane, 1,1,3,3-tetramethylbutyl peroxy-2-ethylhexanoate, tert-amyl peroxy-2-ethylhexanoate, tert-butyl peroxy-2-ethylhexanoate, tert-butyl peroxydiehtlyacetate, tert-butyl peroxyisobutyrate, tert-amylperoxy acetate, tert-butyl peroxy-3,5,5-trimethylhexanoate, tert-amyl peroxybenzoate, tert-butyl peroxyacetate, and tert-butyl peroxybenzoate.

33. The process according to claim 26, wherein the reductor comprises a sulphite source.

34. The process according to claim 33, wherein the sulphite source comprises sodium sulphite, sodium bisulphite, or sodium meta bisulphite.
